(19) United States
(12) Patent Application Publication
(10) Pub. No.: US 2012/0259294 A1
(43) Pub. Date: Oct. 11, 2012

(54) METHOD FOR PRODUCTION OF ELECTRO-SPUN TUBULAR ANASTOMOSIS/REVASCULARIZATION DEVICE FOR TREATMENT OF ISCHEMIC TISSUE

(76) Inventors: Gholam A. PEYMAN, Sun City, AZ (US); Eniko T. ENIKOV, Tucson, AZ (US)

(21) Appl. No.: 13/442,129

(22) Filed: Apr. 9, 2012

Related U.S. Application Data
(60) Provisional application No. 61/516,939, filed on Apr. 11, 2011.

(51) Int. Cl. A61M 25/00; B32B 38/10; B32B 37/02

(52) U.S. Cl. 604/264; 156/250

(57) ABSTRACT

A method of production of a tubular device for treatment of ischemic tissue, comprising dip coating a mandrel in potassium palmitate solution and drying in air, cross-link a tubular device in vapors of a 50% glutar-aldehyde solution, evaporating excess glutar-aldehyde solution by air-drying or vacuum drying the cross-linked tubes in 20-30 mTorr of pressure, scoring and cutting the tubular device into segments, soaking the tubular device in water or water-based solution, placing the tubular device so as to surround the mandrel; and placing a sleeve over the tubular device so as to enable the tubular device to be injected into tissue.

### CROSS REFERENCE TO RELATED APPLICATIONS

This application claims priority to provisional application serial number 61/516,939 filed Apr. 11, 2011, the entire contents of which are incorporated herein by reference.

### GOVERNMENT LICENSE RIGHTS

This invention was made with government support under the US-EU FIPSE mobility grant FRS 309770 grant #P116J080016 from US Department of Education Fund for the Improvement of Postsecondary Education (FIPSE) and by and by grant no. 2008-1767 from the Education, Audiovisual and Culture Executive Agency (EACEA). The government has certain rights in the invention

### BACKGROUND OF THE INVENTION

I. Technical Field

The present invention is related to electro-spinning for producing a variety of porous polymeric substrates.

II. Description of Related Art

Electro-spinning is an emerging technique for producing a variety of porous polymeric substrates. Electro-spinning involves fiber extrusion under high electrostatic fields, which result in fiber stretching and thinning to sub-micron dimensions. Many bio-polymers such as collagen and poly-lactic- co-glycolic acid (PLGA) can used for drug delivery such as VEGF etc. to produce fibrous mats from these materials combined with medication. Upon additional cross-linking process, such as glutaraldehyde treatment, the fibrous mats can be cross-linked and fixed. Subsequently, these mats can be used as tissue templates by seeding them with variety of cell lines and culturing in appropriate media or as drug delivery devices whereby they release therapeutic drugs after implantation in the body. The advantages of natural polymeric drug delivery devices are their bio-absorbability which eliminates adverse foreign-body reaction associated with artificial polymers. Electro-spinning of polymers is a recently emergent technique for producing porous polymeric mats that resemble natural intracellular matrix. By virtue of their porosity and nano-fibrous structure, these mats have been shown to facilitate the incorporation of cells into themselves and create artificial tissues such as vascular or neural anastomoses. Examples of such structures can be found in the teachings of U.S. Pat. No. 6,342,051 B1.

One significant shortcoming of the electro-spinning process is the need for use of conductive collector electrode with a smooth surface finish to prevent adhesion and facilitate the release of the collected fibers. This is particularly challenging when collecting fibers on rotating mandrels where certain adhesion is required to prevent the fiber from flying off the mandrel due to centrifugal and viscous drag forces. While such adhesion is desirable during the fiber collection, it leads to damage and deformation of the template during the release (removal) of the template from the mandrel. Therefore the production of tubular and other closed-surface structures requires a special release layer. Many common mold release materials are unsuitable as they are dielectric and eventually lead to charging of the collector surface and repulsion of the incoming fibers.

### SUMMARY OF THE INVENTION

The present invention relates to a method of production of a tubular device for treatment of ischemic tissue, comprising dip coating a mandrel in potassium palmitate solution and drying in air, cross-link a tubular device in vapors of a 50% glutar-aldehyde solution, evaporating excess glutar-aldehyde solution by air-drying or vacuum drying the cross-linked tubes in 20-30 mTorr of pressure, scoring and cutting the tubular device into segments, soaking the tubular device in water or water-based solution, placing the tubular device so as to surround the mandrel; and placing a sleeve over the tubular device so as to enable the tubular device to be injected into tissue. The device can be loaded with various medications such as vascular endothelial cell factor (VEGF) or anti-VEGFetc. for slow release.

### BRIEF DESCRIPTION OF THE DRAWINGS

FIG. 1 shows examples of tubular prototypes fabricated according to the protocol of this invention;

FIG. 2 shows a method for placement and release of an anastomosis device; and

FIG. 3 shows an embedded (released) anastomosis device in agarose tissue phantom;

### DETAILED DESCRIPTION OF THE INVENTION

According to present invention, we have demonstrated a method for production of a tubular device by the application of a partially conductive film which has sufficient adhesion in dry form (during the collection) but is water soluble and easy to remove during release. Many such partially conductive and water soluble materials exist in the group of salts of fatty acids. For example, we have demonstrated that potassium palmitate CH3(CH2)14COOK can be used to pre-coat a conductive mandrel prior to collecting the electrospun fiber, subsequently, the mat is cross-linked in vapors of glutaraldehyde or other cross-linking agent to permanently fix the shape. Upon exposure to an aqueous environment and even in a dry state, the tubular structure becomes free to slide and be released from the mandrel.

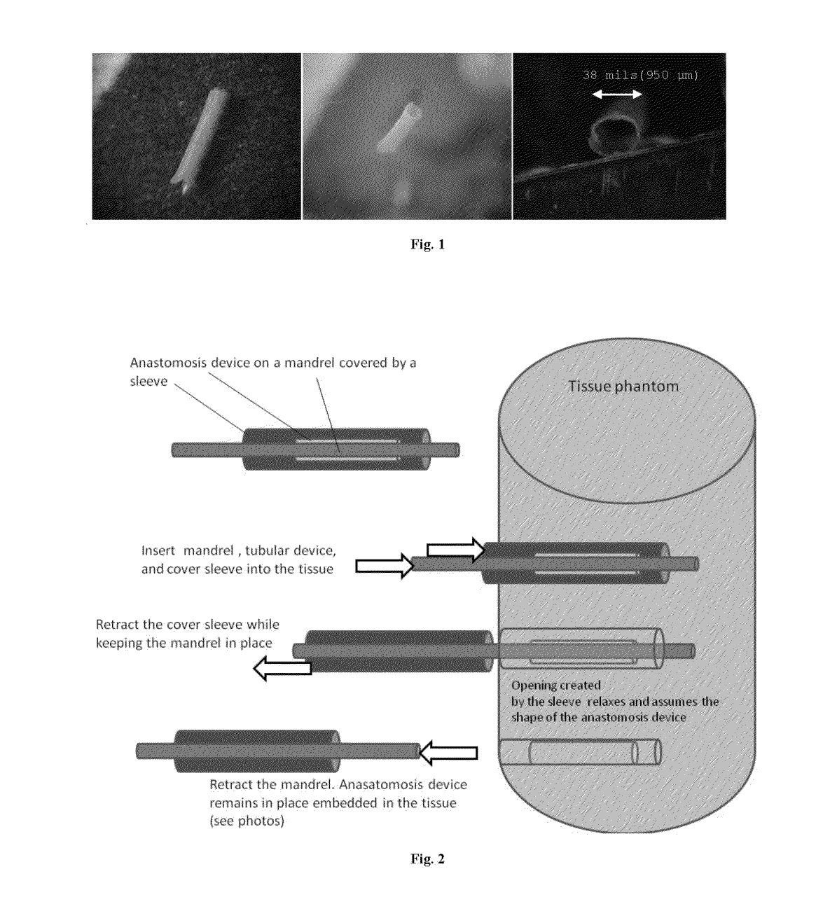

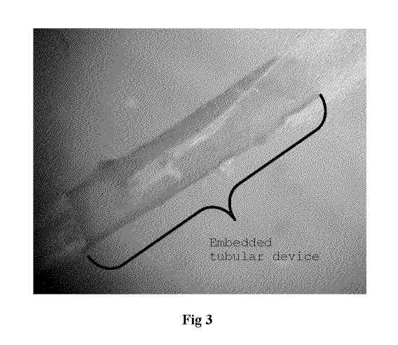

The Following is an Example Process
1. Dip coat the mandrel in 0.5 mol/L potassium palmitate solution and dry in air. Repeat this process several times until an adequate film is formed.
2. Cross-link the resulting tubular device in the vapors of 50% glutar-aldehyde (GLA) solution over period of 8-24 hrs as desired.
3. Allow any excess GLA to evaporate by air-drying or vacuum drying the cross-linked tubes in at 20-30 mTorr of pressure.
4. Score and cut the tubes into segments as desired (see FIG. 1).
5. Soak the tubes in water or water-based solution and load the tube with appropriate medication.
6. Release the resulting tube by sliding off from the mandrel or place a sleeve over the tube and inject into the tissue, then retract the cover sleeve and retract the needle. The tube remains embedded inside the tissue (see FIG. 2.).

We claim:

1. A method of production of a tubular device for treatment of ischemic tissue, comprising:
dip coating a mandrel in potassium palmitate solution and drying in air;
cross-link a tubular device in vapors of a 50% glutar-aldehyde solution;
evaporating excess glutar-aldehyde solution by air-drying or vacuum drying the cross-linked tubes in 20-30 mTorr of pressure;
scoring and cutting the tubular device into segments;
soaking the tubular device in water or water-based solution;
placing the tubular device so as to surround the mandrel; and
placing a sleeve over the tubular device so as to enable the tubular device to be injected into tissue.

2. The method according to claim 1, further comprising
pre-coating the sleeve with a release layer.

3. The method according to claim 2, wherein the release layer is a fatty-acid salt.

4. The method according to claim 3, wherein the fatty acid salt is potassium palmitate CH3(CH2)14COOK.

5. The method according to claim 1, further comprising
collecting the tubular device on a cylindrical rotating electrode.

6. The method according to claim 1, wherein during the soaking, the water based solution contains drugs or signal proteins which are impregnated into the device.

7. The method according to claim 6, wherein the drugs or signal proteins include vascular endothelial growth factor (VEGF).

8. The method according to claim 1, further comprising injecting the tubular device into tissue, so as to treat retinal vein occlusion, or induce revascularization of the ischemic heart.

9. The method according to claim 1, wherein the dip coating a mandrel in potassium palmitate solution includes dip coating the mandrel in a 0.5 mol/L potassium palmitate solution.
